(19) United States
(12) Patent Application Publication
(10) Pub. No.: US 2019/0040447 A1
(43) Pub. Date: Feb. 7, 2019

(54) Assay for compounds having activity against influenza

(71) Applicant: Hoffmann-La Roche Inc., Little Falls, NJ (US)

(72) Inventor: Tianlai SHI, Basel (CH)

(73) Assignee: Hoffmann-La Roche Inc., Little Falls, NJ (US)

(21) Appl. No.: 16/075,241

(22) PCT Filed: Feb. 3, 2017
(86) PCT No.: PCT/EP2017/052386

§ 371 Date: Aug. 3, 2018

(30) Foreign Application Priority Data
Feb. 5, 2016 (EP) 16154496.0

(51) Int. Cl. C12Q 1/48

(52) U.S. Cl. CPC C12Q 1/485; G01N 2500/04; G01N 2333/91275; G01N 2333/11

(57) ABSTRACT

The present invention provides a method for identifying a substance capable of inhibiting at least one activity of an RNA-dependent RNA polymerase (RdRp) comprised in an influenza viral ribonucleoprotein (RNP) complex, which method comprises or consists of: (a) providing an aqueous suspension of purified influenza virus comprising said RNP complex; (b) adding to said suspension of step (a) (i) a nonionic or zwitterionic surfactant; and (ii) a reducing agent; (c) incubating the mixture obtained in step (b) in order to obtain an influenza virus lysate; (d) contacting the influenza virus lysate obtained in step (c) with a test substance under conditions that permit said test substance to interact with said RdRp comprised in said RNP complex, said RNP complex being present in said influenza virus lysate; and (e) determining whether said test substance inhibits said at least one activity of said RdRp.

### FIELD OF THE INVENTION

The present invention provides a method for identifying a substance capable of inhibiting at least one activity of an RNA-dependent RNA polymerase (RdRp) comprised in an influenza viral ribonucleoprotein (RNP) complex, which method comprises or consists of: (a) providing an aqueous suspension of purified influenza virus comprising said RNP complex; (b) adding to said suspension of step (a) (i) a nonionic or zwitterionic surfactant; and (ii) a reducing agent; (c) incubating the mixture obtained in step (b) in order to obtain an influenza virus lysate; (d) contacting the influenza virus lysate obtained in step (c) with a test substance under conditions that permit said test substance to interact with said RdRp comprised in said RNP complex, said RNP complex being present in said influenza virus lysate; and (e) determining whether said test substance inhibits said at least one activity of said RdRp.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### BACKGROUND OF THE INVENTION

Influenza viruses belong to the Orthomyxoviridae family of RNA viruses. Based on antigenic differences of viral nucleocapsid and matrix proteins, influenza viruses are further divided into three types named influenza A, B, and C viruses. All influenza viruses have an envelope, and their genomes are composed of eight or seven single-stranded, negative-sensed RNA segments. These viruses cause respiratory diseases in humans and animals with a significant morbidity and mortality. The influenza pandemic of 1918, Spanish flu, is thought to have killed up to 100 million people. The reassortment of avian flu RNA fragments with circulating human viruses caused the other two pandemics in 1957 H2N2 “Asian influenza” and 1968 H3N2 “Hong Kong influenza”. Now, people around the world face the challenges of influenza from various angles: seasonal influenza epidemics affect about 5-15% of the world's population with an annual mortality ranging from 250,000 to 500,000. Infections of avian flu strains, mostly H5N1, have been reported in many Asian countries. Although no frequent human-to-human spreading has been observed, avian flu infection is serious and associated with a high mortality of up to 60% of infected persons. In 2009, an H1N1 swine flu infection appeared initially in North America and evolved into a new pandemic. Currently, seasonal trivalent influenza vaccines and vaccines specific for H5N1 or swine flu are either available or in the phase of clinical trials. The prophylaxis is an effective method, at least in some populations, for preventing influenza virus infection and its potentially severe complications. However, continuous viral antigenicity shifting and drifting makes future circulating flu strains unpredictable. Furthermore, due to the limitations of mass production of vaccines within a relatively short period of time during a pandemic, other anti-flu approaches such as anti-flu drugs are highly desirable. On the market, there are two types of anti-flu drugs available: neuraminidase inhibitors such as oseltamivir phosphate (Tamilflu) and zanamivir (Relenza); and M2 ion channel blockers such as amantadine and rimantadine. To increase the effectiveness of current anti-flu drugs and prevent or attenuate appearance of drug-resistant viruses, it is invaluable to discover compounds with new mechanisms of anti-influenza action that can be used as a therapeutic or prophylactic agent alone or combined with current anti-flu drugs.

It appears realistic that H5N1 and related highly pathogenic avian influenza viruses could acquire mutations rendering them more easily transmissible between humans. In addition, the new A/H1N1 could become more virulent and only a single point mutation would be enough to confer resistance to oseltamivir (Neumann et al., Nature 2009, 18, 459(7249), 931-939). This has already happened in the case of some seasonal H1N1 strains which have recently been identified (Dharan et al., The Journal of the American Medical Association, 2009, 301(10), 1034-1041; Moscona et al., The New England Journal of Medicine 2009, 360(10), 953-956). The unavoidable delay in generating and deploying a vaccine could in such cases be catastrophically costly in human lives and societal disruption.

In view of the currently elevated risk of infections of pandemic H1N1 swine flu, highly pathogenic H5N1 avian flu, and drug-resistant seasonal flu, the development of new anti-influenza drugs has again become high priority.

In many cases, the development of anti-viral medicament may be facilitated by the availability of structural data of viral proteins. The availability of structural data of influenza virus surface antigen neuraminidase has, e.g. led to the design of improved neuraminidase inhibitors (Von Itzstein et al., Nature 1993, 363, 418-423). Examples of active compounds which have been developed based on such structural data include zanamivir (Glaxo) and oseltamivir (Roche). However, although these medicaments may lead to a reduction of the duration of the disease, there remains an urgent need for improved medicaments which may also be used for curing these diseases.

Adamantane-containing compounds such as amantadine and rimantadine are another example of active compounds which have been used in order to treat influenza. However, they often lead to side effects and have been found to be ineffective in a growing number of cases (Magden et al., Appl. Microbiol. Biotechnol. 2005, 66, 612-621).

More unspecific viral drugs have been used for the treatment of influenza and other virus infections (Eriksson et al., Antimicrob. Agents Chemother. 1977, 11, 946-951), but their use is limited due to side effects (Furuta et al., Antimicrobial Agents and Chemotherapy 2005, 981-986).

Influenza viruses being Orthomyxoviridae, as described above, are negative-sense ssRNA viruses. Other examples of viruses of this group include Arenaviridae, Bunyaviridae, Ophioviridae, Deltavirus, Bornaviridae, Filoviridae, Paramyxoviridae, Rhabdoviridae and Nyamiviridae. These viruses use negative-sense RNA as their genetic material. Single-stranded RNA viruses are classified as positive or negative depending on the sense or polarity of the RNA. Before transcription, the action of an RNA polymerase is necessary to produce positive RNA from the negative viral RNA. The RNA of a negative-sense virus (vRNA) alone is therefore considered non-infectious.

The trimeric viral RNA-dependent RNA polymerase, consisting of polymerase basic protein 1 (PB1), polymerase basic protein 2 (PB2) and polymerase acidic protein (PA) subunits, is responsible for the transcription and replication of the viral RNA genome segments. Structural data of the two key domains of the polymerase, the mRNA cap-binding domain in the PB2 subunit (Guilligay et al., Nature Structural & Molecular Biology 2008, 15(5), 500-506) and the endonuclease-active site in the PA subunit (Dias et al., Nature 2009, 458, 914-918) has become available.

The ribonucleoprotein (RNP) complex represents the minimal transcriptional and replicative machinery of an influenza virus. The polymerase, when comprised in the RNP complex, is also referred to as vRNP enzyme. During replication, the viral RNA polymerase generates a complementary RNA (cRNA) replication intermediate, a full-length complement of the vRNA that serves as a template for the synthesis of new copies of vRNA.

During transcription, the viral RNA polymerase comprised in the RNP complex synthesizes capped and polyadenylated mRNA using 5′ capped RNA primers. This process involves a mechanism called cap snatching. The influenza polymerase uses host cell transcripts (capped pre-mRNAs) as primers for the synthesis of viral transcripts. The nucleoprotein is an essential component of the viral transcriptional machinery. The polymerase complex which is responsible for transcribing the single-stranded negative-sense viral RNA into viral mRNAs and for replicating the viral mRNAs, is thus a promising starting points for developing new classes of compounds which may be used in order to treat influenza (Fodor, Acta virologica 2013, 57, 113-122). This finding is augmented by the fact that the polymerase complex contains a number of functional active sites which are expected to differ to a considerable degree from functional sites present in proteins of cells functioning as hosts for the virus (Magden et al., Appl. Microbiol. Biotechnol. 2005, 66, 612-621). As one example, a substituted 2,6-diketopiperazine has been identified which selectively inhibits the cap-dependent transcriptase of influenza A and B viruses without having an effect on the activities of other polymerases (Tomassini et al., Antimicrob. Agents Chemother. 1996, 40, 1189-1193). In addition, it has been reported that phosphorylated 2′-deoxy-2′-fluoroguanosine reversibly inhibits influenza virus replication in chick embryo cells. While primary and secondary transcription of influenza virus RNA were blocked even at low concentrations of the compound, no inhibition of cell protein synthesis was observed even at high compound concentrations (Tisdale et al., Antimicrob. Agents Chemother. 1995, 39, 2454-2458).

A variety of assay designs are available for the purpose of identifying compounds which are capable of modifying, in many instances inhibiting, the activity of a given target molecule. One of the established distinctions is between cellular assays and biochemical assays. Cellular assays have been described as mimicking closer the in vivo situation, however, they suffer from the drawback that any candidate compound generally has to pass the cell membrane in a first step. Biochemical assays are simpler in that respect. The target, typically a protein, may be presented in enriched or purified form in aqueous solution. In such an assay scenario there is no membrane barrier, however, the conditions may be further remote from the environment in the organism to be subjected to therapy.

Roch et al. (Assay and drug development technologies, 13, 388-506 (2015)) describes an assay for the identification of influenza A virus polymerase inhibitors. The assay employs the entire ribonucleoprotein complex. While being a cell-free assay, the target is in a more complex environment as compared to the pure polymerase. The assay is a transcription assay. The detection scheme is based on hybridization. Radioactivity is not employed.

The technical problem underlying the present invention can be seen in the provision of improved methods for identifying compounds which are capable of interfering with an activity of the viral polymerase of influenza virus. This technical problem is solved by the subject-matter of the claims.

### SUMMARY OF THE INVENTION

The present invention relates to a method for identifying a substance capable of inhibiting at least one activity of an RNA-dependent RNA polymerase (RdRp) comprised in an influenza viral ribonucleoprotein (RNP) complex, which method comprises or consists of: (a) providing an aqueous suspension of purified influenza virus comprising said RNP complex; (b) adding to said suspension of step (a) (i) a nonionic or zwitterionic surfactant; and (ii) a reducing agent; (c) incubating the mixture obtained in step (b) in order to obtain an influenza virus lysate; (d) contacting the influenza virus lysate obtained in step (c) with a test substance under conditions that permit said test substance to interact with said RdRp comprised in said RNP complex, said RNP complex being present in said influenza virus lysate; and (e) determining whether said test substance inhibits said at least one activity of said RdRp.

Accordingly, provided is a screening method. As will be discussed in more detail below, the method is apt for an implementation in a high throughput format. Also, it permits the determination of IC₅₀ values of active substances.

The term “inhibiting” has its art-established meaning in the context of reducing the activity of an enzyme or a binding molecule. The term “activity” includes enzymatic activity and extends to the capability to bind a (preferably cognate) ligand. “Inhibiting” refers to a reduction of activity by a factor of at least 10⁻¹, at least 10⁻², at least 10⁻³, at least 10⁻⁴, at least 10⁻⁵, at least 10⁻⁶, at least 10⁻⁷, at least 10⁻⁸, at least 10⁻⁹, and/or below the detection limit.

### DETAILED DESCRIPTION OF THE INVENTION

Influenza viruses have a segmented negative sense ssRNA genome. During the viral life cycle, an RNA-dependent RNA polymerase is needed which provides replication and transcription activity. The polymerase is virus encoded. Polymerase molecules are comprised in each ribonucleoprotein complex, and a plurality of ribonucleoprotein complexes is enclosed by a viral envelope. Influenza virus, the ribonucleoprotein complex and the viral RNA-dependent RNA polymerase are discussed herein above and pertinent references are cited. The definition of these terms as provided herein above are definitions in accordance with the present invention. The method in accordance with the present invention employs the RNA-dependent RNA polymerase in its form as bound within the ribonucleoprotein complex. A viral envelope is not present.

In accordance with step (a) an aqueous suspension of RNPs is provided. The suspension is preferably in HEPES buffer. The term “purified” as used in conjunction with influenza viruses means that no polymerases other than the viral RdRp are present. In particular, no polymerases of the host system used for preparation of influenza viruses are present. A preferred host system are chicken eggs. Accordingly, and to the extent said host system is used, chicken polymerases are absent from said purified influenza viruses.

A preferred virus purification procedure is sucrose gradient purification: The infectious allantoic fluid (AF) is harvested from each egg. The AF is then concentrated via tangential flow filtration (TFF), and the virus is pelleted via centrifugation. The pellet is resuspended in a Hepes-buffered saline and is placed on a sucrose step gradient and centrifuged. The interface band is removed and washed in buffer to remove sucrose. The pellet is then resuspended in the Hepes-buffered saline and a colorimetric protein assay is performed on the bulk material. The material is diluted, preferably to 2 mg/ml in buffer, and aliquoted for standard inventoried antigen. Inventoried product is preferably tested for hemagglutination, an EID50 titer (embryo infectious dosage @ 50%), and protein concentration. The hemagglutination test indicates the concentration of virus particle. Preferred influenza viruses are disclosed further below.

Step (b) provides for adding to the suspension of step (a) at least two types of agents, each of which agents may be a single compound or a mixture of two or more compounds.

Agent (i) is a non-ionic or zwitterionic surfactant. Preferred surfactants in accordance with the present invention are non-denaturing.

The term “non-denaturing” has its art-established meaning and refers to surfactants which do not cause unfolding, aggregation and/or loss of function of proteins. Whether or not the given surfactant is non-denaturing can be determined by the skilled person without further ado, in particular when provided with the teaching of the present invention. To explain further, the method in accordance with the invention, when performed in the absence of any test substance, is an assay for polymerase activity. To the extent a denaturing surfactant would be used, polymerase activity would be decreased or abolished. Non-denaturing surfactants are those which cause less than 50% loss of transcription activity of the RdRp when assayed under the conditions given in the examples enclosed herewith and compared to the use of Triton® X-100 as a surfactant.

The preferred non-ionic surfactants and zwitterionic surfactants as given in Tables 1 and 2 below, respectively, fulfill the requirement of being non-denaturing.

Agent (ii) is a reducing agent. Accordingly, a compound with an electron donating functional group may be employed as agent (ii). The reducing agent is capable of donating an electron to another chemical species in a redox reaction. Said chemical species includes disulphide bonds as they occur, e.g. in proteins. In other words, a reducing agent in accordance with the invention prevents intramolecular and intermolecular disulfide bonds between cysteine residues of proteins from forming.

Further agents may, but do not have to be added at step (b). A preferred agent to be added at step (b) is a stabilizer; see further below. A further preferred agent to be added is an RNAse inhibitor. Further preferred agents to be added are those described in Example 1.

Subsequently, the mixture obtained in step (b) is incubated to yield an influenza virus lysate. Suitable conditions, especially in terms of time and temperature are known in the art or can be determined by the skilled person without further ado. Preferred conditions are given further below.

In accordance with the present invention, the influenza virus lysate obtained in step (c) is not subjected to purification. Prior art protocols, including those which employ the RNP complex as a target for screening, generally purify the RNP complexes from the influenza virus lysate. There is a belief in the art that such purification would be indispensable and/or better results would be obtained in the screen when such purification is effected. The present inventors surprisingly found that the contrary is true. Dispensing with purifying the RNP complexes from the influenza virus lysate yields better results, introduces less bias and avoids loss of material. More specifically, and as compared to art-established protocols (see, e.g., Klumpp et al., Meth. Enzymol. 342, 451 (2001)), the method of the present invention is considerably faster, yields more enzyme, and, importantly, the obtained material exhibits the same quality, in particular with regard to screening of compounds.

In step (d), the influenza virus lysate obtained in step (c) is brought into contact with a test substance. Said contacting is to be effected under conditions that permit said test substance to interact with the RNA-dependent RNA polymerase. Suitable conditions can be determined by the skilled person without further ado. Preferred conditions include buffered solutions. A preferred buffer is HEPES, preferably at a pH=7.5. Preferably, an RNAse inhibitor is present. Furthermore, conditions preferably include a magnesium salt and/or a manganese salt, e.g. Mg(OAc)₂ and/or Mn(OAc)₂. The test substance may be added as a solution in an organic solvent or as an aqueous solution. Suitable organic solvents include those commonly used for compound libraries, for example DMSO. Examples 2 and 3 describe particularly preferred conditions.

Step (e) provides for determining whether a given test substance is a “hit” in the screen, i.e. a substance capable of inhibiting at least one activity of the RdRp. Preferred implementations of said determining depend on the particular activity of the RdRp to be assessed. Preferred implementations of step (e) are detailed further below.

The term “test substance” is not particularly limited. It is understood that the test substance is soluble under the conditions of step (d) of the method of the invention. Preferably, a test substance or libraries of test substances are chosen such that a capability of inhibiting one RdRp activity can be expected or that one or more members of said libraries can be expected to exhibit such capability. Accordingly, there is a formal distinction between a “test substance” and a “substance capable of inhibiting at least one activity of an RdRp”: a test substance may have such capability, but does not have to. The method of the invention provides for identifying those substances among the test substances which are indeed capable of inhibiting at least one activity of an RdRp.

Preferred test substances are small organic molecules, preferably having a molecular weight below 1,000 Da, below 900 Da, below 700 Da, below 600 Da or below 500 Da.

In a preferred embodiment of the method of the invention, said activity is selected from transcription, Cap-binding, endonuclease activity, replication and polymerase activity.

The key activity of the RNA-dependent RNA polymerase during the viral life cycle is replication, i.e. the synthesis of copies of the viral genome, and transcription, i.e. the synthesis of viral mRNA transcripts. In either case, the products are polynucleotides.

Therefore, the use of labeled nucleotides, preferably radioactively labeled nucleotides, allows for the incorporation of radioactive label into said nucleotides.

During replication, the polymerase activity comprised in the RdRp is involved. During transcription, the Cap-binding activity, the endonuclease activity, and the polymerase activity are involved.

Accordingly, in a further preferred embodiment, said determining in step (e) comprises or consists of quantifying transcription and replication. In the alternative, only transcription or only replication is determined or quantified, respectively.

In a preferred embodiment, step (e) comprises (e1) adding at least one radioactively labelled nucleotide; (e2) allowing transcription and/or replication to occur so that the radioactively labelled nucleotide is incorporated into a product of transcription or replication; (e3) precipitating the product of step (e2) on a filter; and (e4) quantifying the radioactivity of the product retained on the filter; preferably by scintillation counting.

To the extent the polynucleotides synthesized by the RdRp are RNA, said radioactively labeled nucleotide is a ribonucleotide. It may be any one of ATP, CTP, UTP and GTP. Even though less preferred, also two or more types of radioactively labeled ribonucleotides may be used. Typically one radioactively labeled nucleotide is used. A mixture of radioactively labeled and non-labeled nucleotides of the same type, for example GTP, may be used; see Examples 2 and 3.

Allowing transcription and/or replication to occur in accordance with step (e2) includes the provision of conditions where these processes can occur. These conditions are known in the art and can be established by the skilled person without further ado. Further guidance is given below.

It is understood that “allowing transcription and/or replication to occur” also includes the provision of all four ribonucleotides in step (e1), namely ATP, CTP, UTP and GTP, wherein one or more thereof may be radioactively labeled as described above. To the extent step (e) comprises quantifying transcription, a capped mRNA is added in step (e1). To the extent step (e) comprises quantifying replication, a primer such as pppApG is added in step (e1).

In the following, a preferred implementation of the transcription assay in accordance with the invention is provided. This in vitro assay permits to identify inhibitors of Cap-binding, endonuclease and polymerase activities of the Influenza A or B virus. Influenza ribonucleoprotein complexes (RNPs) are responsible for the transcription and replication of viral genomic negative strain RNA to positive strain mRNA and positive strain cRNA respectively. The transcription is initiated by the “cap-snatching” mechanism which consists of two steps: The cap-binding of cellular mRNA by the PB2 subunit and the cleavage of the capped RNA by the PA subunit. The resulting typically 9-13 nucleotide long, capped RNA oligo serves as a primer for the subsequent synthesis of viral mRNA by the polymerase subunit PB1. During the mRNA synthesis, radiolabeled nucleotide will be incorporated into the mRNA product, which will be captured on a specific filter plate by precipitation, e.g. with trichloroacetic acid (TCA). The efficiency of nucleotide incorporation is then determined by quantifying the captured mRNA on the filter plate, preferably by scintillation counting. A higher rate of mRNA synthesis leads to higher signals. Due to the essential involvement of cap-binding and cleavage reaction prior to polymerization of mRNA, it is possible to inhibit transcription by either blocking the endonuclease active site of PA or the cap-binding site of PB2. IC₅₀ values of both endonuclease and cap-binding inhibitors may also be determined.

In the following, a description of a preferred replication assay of the invention is provided. This in vitro assay permits to identify inhibitors targeting polymerase activities of the Influenza A virus, preferably Influenza A. Influenza ribonucleoprotein complexes (RNPs) are responsible not only for the transcription of negative-sense viral genomic RNA (vRNA) to positive-sense mRNA, but also for the replication of full-length complementary genomic RNA (cRNA). A pppApG dinucleotide is provided to the RNPs to initiate the cRNA synthesis and during the elongation process, radiolabeled nucleotide will be incorporated into the cRNA product, which will be captured on a specific filter plate by TCA precipitation. The efficiency of nucleotide incorporation is then determined, preferably by scintillation counting of captured cRNA, preferably on a filter plate. A higher rate of cRNA synthesis leads to higher signals. Due to the essential involvement of polymerase subunit for the polymerization of cRNA, it is possible to inhibit replication by either directly blocking the polymerase active site of PB1 or by preventing the conformational changes of RNP that is required for the realignment of polymerase complex on the vRNA template. This assay can be used to determine IC₅₀ values of replication inhibitors.

In accordance with step (e3), the products of step (e2) are precipitated on a filter.

Preferably, said filter is a millipore filter. Preferred pore sizes are below 10 μm, below 5 μm or below 2 μm. Especially preferred are pore sizes of 1.2 μm and 0.65 μm. The filter membrane may be a glass filter. Preferably, a vacuum manifold is used. Multiwell plates such as 96 or 384 well plates which are compatible with TCA precipitation are preferred.

Step (e4) provides for quantifying the radioactivity retained on the filter, thereby quantifying the radioactively labeled polynucleotides which in turn are products of the RdRp. A preferred method of quantifying radioactivity is scintillation counting.

In the following, a particularly preferred implementation of the filtering process (steps (e3) and (e4)) is provided.

Polynucleotides, in particular synthesized mRNA products from the transcription reaction or cRNA products from the replication reaction are precipitated on the filter plate using 5-30% TCA), preferably 10% TCA at <25° C., preferably at 4° C., for 5 min to 1 h, preferably 35 minutes and followed by washing multiple times with >5% TCA and >50% ethanol, preferably by three times wash with 10% TCA and 1 time with 70% ethanol on the vacuum manifold. After complete air dry of the filter plate, scintillation cocktail, preferably Microsint 20 solution is added to the wells and scintillation counting is performed on a scintillation counter, preferably TopCount equipment. Dose-response curves are analyzed using 4-parameter curve fitting methods. The concentration of test compound resulting in 50% inhibition to that of the control wells is reported as IC₅₀.

Generally speaking, the filtering process of steps (e3) and (e4) advantageously neither involves chromatographic or electrophoretic separation nor it is based on hybridization. Therefore, the filtering process renders the method of the invention particularly amenable for high throughput. Compared to autoradiography and its associated quantification after electrophoretic separation as an art-established method, the invention here using a filtering step and subsequent scintillation counting is a much faster and less labor intensive process for sample separation, signal visualization as well as quantification.

In accordance with the main embodiment, the influenza virus lysate obtained in step (c) is brought into contact with a test substance. Accordingly, it is understood that there is no intervening step between steps (c) and (d), especially no intervening purification or separation. As noted above, the present inventors surprisingly found that purification of the RNP complexes is dispensable. In other words, said method does not involve separation of RNP complexes from other viral components present in the viral lysate and/or said method does not involve purification of said influenza virus lysate of step (c) before step (d).

In a further preferred embodiment of the method of the invention, said non-ionic surfactant is selected from polyoxyethylene glycol alkyl ethers; polyoxypropylene glycol alkyl ethers; glucoside alkyl ethers; polyoxyethylene glycol octylphenol ethers; polyoxyethylene glycol alkylphenol ethers; glycerol alkyl esters; polyoxyethylene glycol sorbitan alkyl esters; and sorbitan alkyl esters or a mixture of at least two of these compounds; preferably polyoxyethylene glycol octylphenol ethers; and/or said zwitterionic surfactant is selected from sultaines; betaines; and sulfobetaines; or a mixture of at least two of these compounds. Preferably said non-ionic surfactant is selected from sulfobetaines.

In a preferred embodiment, said reducing agent is selected from dithiothreitol, 2-mercaptoethanol, tris(2-carboxyethyl)phosphine HCl (TCEP), cysteine, and 2-mercaptoethylamine, or a mixture of at least two of these compounds. Preferably said reducing agent is a mixture of dithiothreitol and 2-mercapoethanol.

To the extent mixtures are used, these may be mixtures of compounds of different classes, for example one polyoxyethylene glycol octylphenol ether and one polyoxyethylene glycol alkyl ether, or these may be mixtures of two or more compounds from the same compound class, for example two or more polyoxyethylene glycol octylphenol ethers. Also, it is possible to use one or more non-ionic surfactants in a mixture with one or more zwitterionic surfactants.

As noted above, in the present invention typically use is made of non-denaturing surfactants. Preferred non-ionic and zwitterionic surfactants are given in Tables 1 and 2 below, respectively.

TABLE 1

Triton ® X-100
Polyethylenglycol-[4-(1,1,3,3-tetramethylbutyl)phenyl]-ether
C₁₄H₂₂O(C₂H₄O)ₙ, n = 9-10
Triton ® X-114
Polyethylenglycol-[4-(1,1,3,3-tetramethylbutyl)phenyl]-ether
C₁₄H₂₂O(C₂H₄O)ₙ, n = 7-8
Brij ® 35
Polyoxyethylene (23) lauryl ether
Brij ® 58
Polyoxyethylene (20) cetyl ether
Nonidet ® P-40 Substitute
Nonylphenyl polyethylene glycol
Octyl β Glucoside
MEGA 8
Octanoyl-N-methylglucamide
MEGA 9
Nonaoyl-N-methylglucamide
MEGA 10
Decanoyl-N-methylglucamide
BigCHAP
N,N-Bis[3-(D-gluconamido)propyl]cholamide
Deoxy Big CHAP
N,N-Bis[3-(D-gluconamido)propyl]deoxycholamide

TABLE 2
CHAPS
3-[(3-Cholamidopropyl)dimethylammonio]-1-propanesulfonate
CHAPSO
3-[(3-Cholamidopropyl)dimethylammonio]-2-hydroxy-1-propanesulfonate
Sulfobetaine 3-10 (SB 3-10)
N-Decyl-N,N-dimethyl-3-ammonio-1-propanesulfonate
Sulfobetaine 3-12 (SB 3-12)
N-Dodecyl-N,N-dimethyl-3-ammonio-1-propanesulfonate
Sulfobetaine 3-14 (SB 3-14)
N-Tetradecyl-N,N-dimethyl-3-ammonio-1-propanesulfonate
ASB-14
Amidosulfobetaine-14
ASB-16
Amidosulfobetaine-16
ASB-C80
4-n-Octylbenzoylamido-propyl-dimethylammonio sulfobetaine
Non-Detergent Sulfobetaine (ND SB) 201
3-(1-Pyridino)-1-propane sulfonate

Preferred reducing agents are provided in Table 3.

TABLE 3
Reducing agent

| | |
|---|---|
| 2-Mercaptoethanol | 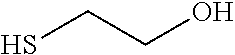 |
| | 2-Mercaptoethanol |
| | (2-ME or βME) |
| | MW 78.13 |
| 2-Mercaptoethylamine- HCl | 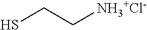 |
| | 2-Mercaptoethylamine |
| | hydrochloride (2-MEA) |
| | MW 113.61 |
| Cysteine-HCl | 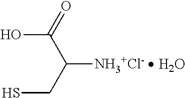 |
| | Cysteine · HCl · H₂O |
| | MW 175.63 |
| Dithiothreitol | 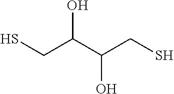 |
| | Dithiothreitol (DTT) |
| | MW 154.25 |
| TCEP-HCl | 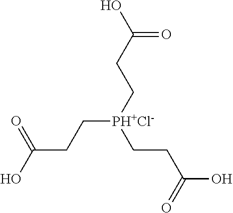 |
| | TCEP · HCl |
| | MW 286.65 |

In a further preferred embodiment, in step (b) furthermore (iii) a stabilizer, preferably a cryoprotectant is added, said cryoprotectant preferably being glycerol, ethylene glycol or a mixture thereof.

A cryoprotectant is useful if it is intended to freeze the influenza virus lysate for storage.

In a further preferred embodiment, the resulting concentration in step (b) of the surfactant is about 0.05 to about 5% (w/v), preferably about 0.1 to about 2% (w/v). Particularly preferred is a concentration of the surfactant of about 1% (w/v). Further envisaged concentrations are about 3% and about 4% (w/v).

In a further preferred embodiment, the resulting concentration in step (b) of the reducing agent is about 0.5 to about 100 mM, preferably about 10 to about 25 mM. Particularly preferred is a concentration of the reducing agent of about 20 mM.

In a further preferred embodiment, the resulting concentration in step (b) of the stabilizer, to the extent present, is about 0.5 to about 60% (w/v), preferably about 1 to about 50% (w/v), and more preferably about 2 to about 25% (w/v). Particularly preferred is a concentration of the stabilizer of about 5% (w/v). Further envisaged concentrations are about 10%, about 15% and about 20% (w/v).

In a further preferred embodiment (i), (ii) and, to the extent present, (iii) are comprised in a solution, preferably an aqueous solution, said solution to be added in step (b) to said suspension. Preferred is a buffered aqueous solution. A preferred buffer is Tris.

In the alternative, only two of (i), (ii) and (iii) may be comprised in a single solution, the third component, to the extent present, being in a separate solution. Furthermore, it is also envisaged to use three distinct solutions, each comprising (i), (ii) and (iii), respectively.

In a further preferred embodiment, said incubating in step (c) is for about 5 to about 180 min, preferably for about 15 to about 60 min, and more preferably for about 30 min.

In a further preferred embodiment, said incubating in step (c) is at a temperature of about 5 to about 35° C., preferably about 15 to about 30° C., and more preferably at about 25° C.

Accordingly, it is especially preferred to perform incubating for about 30 minutes at about 25° C.

It is preferred, that said RNP complex is a native RNP complex obtained from a virus. Alternatively, it may also be a mutant or homologue thereof. Such mutant or homologue may also be obtained from a virus or may be artificially prepared, for example by genetic engineered methods. To the extent mutants or homologues are to be used for the method of the invention, it is understood that the RNA-dependent RNA polymerase comprised in the mutant or homologous RNP complex exhibits at least one, preferably all of Cap-binding, endonuclease and polymerase activity.

In a further preferred embodiment, the influenza virus is influenza A virus or influenza B virus.

Particularly preferred is the influenza A virus which is Influenza A/PR/8/34. Influenza A/PR/8/34 is available from Charles River Vaccine Services (Wilmington, Mass., USA), Cat. No. 10100374. Particularly preferred is the influenza B virus which is Influenza B\Lee\40). Influenza B\Lee\40 is available from Charles River Vaccine Services (Wilmington, Mass., USA), Cat. No. 10100379.

In a further preferred embodiment said method is a high-throughput method.

The term “high-throughput” is well-known in the art. In the course of a high-throughput method, at least 100 test compounds, preferably at least 1,000 test compounds, and more preferably at least 10,000 test substances are tested. Preferably, at least 10000 test substances per day are tested.

High-throughput assays generally may be performed in wells of microtiter plates, wherein each plate may contain 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to said activity.

In a further preferred embodiment, the method of the invention further comprises (f) determining the concentration of said test substance which inhibits 50% of said activity (IC₅₀).

Advantageously, the method of the invention permits the determination of IC₅₀ values of test compounds inhibiting at least one activity of said RdRp.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

### EXAMPLES

The examples illustrate the invention. However, the scope of the application is not intended to be limited thereto.

### Example 1

Preparation of Virus Lysate Containing Native Influenza vRNP Complex

Influenza purified virus (Influenza A/PR/8/34, Influenza B\Lee\40) was obtained from Charles River Laboratories International Inc. as a suspension in HEPES buffer. Virons were disrupted by incubation with an equal volume of 2% Trition X-100 for 30 minutes at room temperature in a buffer containing 40 mM Tris-HCl, pH 8, 5 mM MgCl₂, 200 mM KCl, 100 mM NaCl, 10 mM dithiothreitol [DTT], 5% glycerol, 40 U/ml RNAse inhibitor, 10 mM 2-mercaptoethanol, and 2 mg/ml lysolechithin. The virus lysate was aliquoted and stored at −80° C. in aliquots.

### Example 2

### Influenza A or B Filter RNP Transcription Assay

Virus lysate (H1N1 Influenza strain A/PR/8/34, Charles River, Cat #10100374; Influenza B\Lee\40, Charles River, Cat#10100379) was pre-incubated with test substances for 30 min at 30° C. in the reaction buffer containing 24 mM HEPES (pH 7.5), 118 mM NaOAc, 1 mM Mg(OAc)₂, 0.1 mM Mn(OAc)₂, 0.1 mM EDTA, 2 mM DTT, 0.3 U RNase inhibitor (Riboguard), 70 mM ATP/CTP/UTP, 14 mM GTP and 0.175 μCi ³³P-GTP. Then capped RNA substrate was added to the reaction mixture at 0.07 pM (5⁷m⁷G-ppp-GAA UAC UCA AGC UAU GCA UC-3′, 5′-triphosphorylated RNA was purchased from Fidelity Systems and the capping reaction was performed using the ScriptCap Capping System from CellScript). The Cap-snatching and subsequent mRNA synthesis reactions were performed for 90 min at 30° C. before the reactions were terminated by EDTA addition. Synthesized mRNA products were precipitated on the filter plate (Millipore) and processed as described in Example 4.

### Example 3

### Influenza RNP-Based Replication Assay

The concentrations refer to final concentrations unless mentioned otherwise. Test substances were serially diluted 4 fold in 40% DMSO and 2 μl of diluted test substance was added to 17 μl reaction mixture containing 0.35 nM vRNP enzyme, preferably Influenza A vRNP enzyme, 20 mM HEPES (pH 7.5), 100 mM NaOAc, 1 mM Mg(OAc)₂, 0.1 mM Mn(OAc)₂, 0.1 mM EDTA, 2 mM DTT, 0.25 U RNase inhibitor (Epicentre), 70 pM ATP/CTP/UTP, 1.4 pM GTP and 0.175 μCi ³³P-GTP for 30 minutes at 30° C. pppApG dinucleotide was added to the reaction mixture at 75 pM as final concentration. Reactions were performed for 3 hours at 30° C. and then stopped by adding EDTA to a final concentration of 56 mM. Synthesized cRNA products from the replication reaction were precipitated on the filter plate (Millipore) and processed as described in Example 4.

### Example 4

### Filtration Step for Both Filter RNP Transcription & Replication Assays

Synthesized mRNA products from the transcription reaction or cRNA products from the replication reaction were precipitated on the filter plate using 10% TCA at 4° C. for 35 minutes and followed by three times wash with 10% TCA and 1 time with 70% ethanol on the vacuum manifold. After complete air dry of the filter plate, Microsint 20 solution was added to the wells and scintillation counting was performed on the TopCount equipment. Dose-response curves were analyzed using 4-parameter curve fitting methods. The concentration of test compound resulting in 50% inhibition to that of the control wells were reported as IC₅₀.

### Example 5

### HTS Screen and Results

Using the method of the invention, in particular as disclosed in the preceding examples, a high-throughput screening campaign has been performed. Numerous hits have been found; see, e.g., PCT application entitled “Pyrimidine and pyridine derivatives and their use in the treatment, amelioration or prevention of a viral disease” and filed by the same assignee in the SIPO on the same day as the present application.

Exemplary hits include:

### (−)-(1R,2S,4R,5S,6S,7S)-7-((5-fluoro-2-(5-fluoro-1H-pyrazolo[3,4-b]pyridin-3-yl)pyrimidin-4-yl)amino)tricyclo[3.2.2.02,4]nonane-6-carboxylic Acid

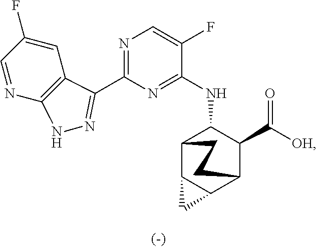

and

### (−)-N-[(1R,2S,4R,5S)-4-[[5-Fluoro-2-(5-fluoro-1H-pyrazolo[3,4-b]pyridin-3-yl)pyrimidin-4-yl]amino]-2-bicyclo[3.1.0]hexanyl]benzamide

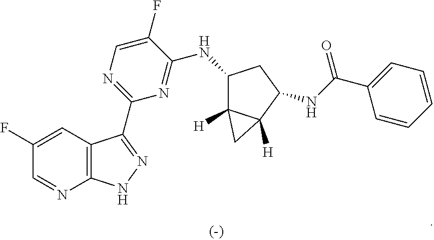 [SMILES: O=C(N[C@H]1C[C@@H](Nc2nc(-c3n[nH]c4ncc(F)cc34)ncc2F)[C@H]2C[C@H]21)c1ccccc1]

These compounds were determined to have IC₅₀ values of 0.2 nM and 0.112 pM, respectively.

1-14. (canceled)

15. A method for identifying a substance capable of inhibiting at least one activity of an RNA-dependent RNA polymerase (RdRp) comprised in an influenza viral ribonucleoprotein (RNP) complex, the method comprising:
(a) providing an aqueous suspension of purified influenza virus comprising said RNP complex;
(b) adding to said suspension of step (a)
(i) a nonionic or zwitterionic surfactant; and
(ii) a reducing agent;
(c) incubating the mixture obtained in step (b) in order to obtain an influenza virus lysate;
(d) contacting the influenza virus lysate obtained in step (c) with a test substance under conditions that permit said test substance to interact with said RdRp comprised in said RNP complex, said RNP complex being present in said influenza virus lysate; and
(e) determining whether said test substance inhibits said at least one activity of said RdRp.

16. The method of claim 15, wherein said activity is selected from transcription, Cap-binding, endonuclease activity, replication and polymerase activity.

17. The method according to claim 15, wherein said determining in step (e) comprises quantifying transcription and/or replication.

18. The method according to claim 16, wherein step (e) comprises
(e1) adding at least one radioactively labelled nucleotide;
(e2) allowing transcription and/or replication to occur so that the radioactively labelled nucleotide is incorporated into a product of transcription or replication;
(e3) precipitating the product of step (e2) on a filter; and
(e4) quantifying the radioactivity of the product retained on the filter; preferably by scintillation counting.

19. The method according to claim 15, wherein said method does not involve separation of RNP complexes from other viral components present in the viral lysate and/or said method does not involve purification of said influenza virus lysate of step (c) before step (d).

20. The method according to claim 15, wherein
(i) said non-ionic surfactant is selected from polyoxyethylene glycol alkyl ethers; polyoxypropylene glycol alkyl ethers; glucoside alkyl ethers; polyoxyethylene glycol octylphenol ethers; polyoxyethylene glycol alkylphenol ethers; glycerol alkyl esters; polyoxyethylene glycol sorbitan alkyl esters; and sorbitan alkyl esters or a mixture of at least two of these compounds; and
(ii) said reducing agent is selected from dithiothreitol, 2-mercaptoethanol, tris(2-carboxyethyl)phosphine HCl (TCEP), cysteine, and 2-mercaptoethylamine, or a mixture of at least two of these compounds.

21. The method according to claim 15, wherein said nonionic surfactant is selected from polyoxyethylene glycol octylphenol ethers.

22. The method according to claim 15, wherein said zwitterionic surfactant is selected from sultaines; betaines; and sulfobetaines; a mixture of at least two of these compounds; or a mixture of least two compounds wherein one compound is sulfobetaines.

23. The method according to claim 15, wherein said reducing agent is a mixture of dithiothreitol and 2-mercapotethanol.

24. The method according to claim 15, wherein in step (b) further comprising
(iii) a stabilizer comprising a cryoprotectant, wherein said cryoprotectant is selected from glycerol, ethylene glycol or a mixture thereof.

25. The method according to claim 15, wherein the resulting concentration in step (b) of:
(i) the surfactant is about 0.05 to about 5% (w/v); and
(ii) the reducing agent is about 0.5 to about 100 mM.

26. The method according to claim 24, wherein the resulting concentration in step (b) of
(i) the surfactant is about 0.05 to about 5% (w/v);
(ii) the reducing agent is about 0.5 to about 100 mM; and
(iii) the stabilizer is about 0.5 to about 60% (w/v).

27. The method of claim 15, wherein (i) and (ii) are comprised in one or more solutions, said solutions to be added in step (b) to said suspension.

28. The method of claim 27, wherein the solution is an aqueous solution.

29. The method of claim 24, wherein (i), (ii) and, (iii) are comprised in one or more solutions, said solutions to be added in step (b) to said suspension.

30. The method according to claim 15, wherein said incubating in step (c) is for about 5 to about 180 min, about 15 to about 60 min, or about 30 min.

31. The method according to claim 15, wherein said incubating in step (c) is at a temperature of about 5 to about 35° C., about 15 to about 30° C., or at about 25° C.

32. The method of claim 15, wherein the influenza virus is influenza A virus or influenza B virus.

33. The method of claim 15, wherein said method is a high-throughput method.

34. The method of claim 15 further comprising:
(f) determining the concentration of said test substance which inhibits 50% of said activity.


SEQUENCE LISTING (1 sequence)

SEQ ID NO: 1 - RNA, 20 nt, Artificial Sequence
  RNA substrate
  misc_feature 1: /note=“The 5′ G carries a cap (7-Me G-ppp)”
gaauacucaagcuaugcauc